(19) United States
(12) Patent Application Publication
(10) Pub. No.: US 2012/0215308 A1
(43) Pub. Date: Aug. 23, 2012

(54) POWER TRANSFER IN A MEDICAL IMPLANT

(76) Inventors: Mathew Ross Markey, Oatley (AU); Andrew Saldanha, St. Ives (AU); Torsten Lehmann, Earlwood (AU)

(21) Appl. No.: 13/030,028

(22) Filed: Feb. 17, 2011

(51) Int. Cl. A61F 2/18; A61F 2/02

(52) U.S. Cl. 623/10; 623/11.11

(57) ABSTRACT

A medical implant comprising a power source configured to generate a direct current (DC) signal, a first interface configured to convert the DC signal to an alternating current (AC) signal; and a DC power storage device. The implant also comprises a second interface configured to convert the AC signal received from the first interface via two interface connection lines to a DC signal, and configured to provide the DC signal to the power storage device via two storage connection lines, and wherein the second interface includes: at least one switch disposed between the two module connection lines and the two storage connection lines; and at least one switch controller configured to control the at least one switch to prevent the power storage device from being electrically connected to an opposite polarity of the power source.

### BACKGROUND

1. Field of the Invention

The present disclosure generally relates to medical implants, and more particularly, to power transfer in a medical implant.

2. Related Art

Medical implants, sometimes referred to as implantable medical devices, require some form of power to operate and perform their intended functions. This power may be provided from a power source external to the implant recipient, or by an internal power source such as, for example, a battery implanted in the recipient.

In some medical implants, it is necessary to transfer power between components or modules of the implant. In medical implants, power storage and power used to drive operational circuits is usually in direct current (DC) form. When transfer or transmission of electrical power is performed within the body of a recipient of the medical implant, contact with the tissue of the recipient should be avoided, or at least minimized, to prevent damage to the tissue.

One particular medical device in which power transfer may be used is a hearing prosthesis, such as a cochlear implant. A cochlear implant allows for electrical stimulating signals to be applied directly to the auditory nerve fibres of the patient, allowing the brain to perceive a hearing sensation approximating the natural hearing sensation. These stimulating signals are applied by an array of electrodes implanted into the patient's cochlea.

### SUMMARY

According to one aspect of the present invention, a medical implant is disclosed. The medical implant comprises a power source configured to generate a direct current (DC) signal; and a first interface configured to convert the DC signal to an alternating current (AC) signal; a DC power storage device; a second interface configured to convert the AC signal received from the first interface via two interface connection lines to a DC signal, and configured to provide the DC signal to the power storage device via two storage connection lines, and wherein the second interface includes: at least one switch disposed between the two module connection lines and the two storage connection lines; and at least one switch controller configured to control the at least one switch to prevent the power storage device from being electrically connected to an opposite polarity of the power source.

According to another aspect of the present invention, an internal component of a medical implant system is disclosed. The internal component comprises a DC power source; and a DC/AC converter adapted to convert a DC signal from the power source to an AC signal; a DC power storage device; and a circuit connected to the DC/AC converter via two interface connection lines, and connected to the power storage device by two storage lines, wherein the circuit is configured to convert the AC signal and a DC signal, the circuit comprising: at least one switch disposed between the two module connection lines and the two storage connection lines; and at least one switch controller for controlling the at least one switch such to prevent the power storage device from being electrically connected to an opposite polarity of the power source.

According to another aspect of the present invention, a method for transferring power from a power source to a power storage device of a medical implant is disclosed. The method comprises converting, with a first interface, a direct current (DC) signal from the power source into an alternating current (AC) signal; providing the AC signal to a second interface via two interface connection lines; converting, with the second interface, the AC signal to a DC signal for deliver to a power storage device via two storage connection lines, wherein the second interface comprises at least one switch disposed between the interface connection lines and the storage connection lines; and causing the at least one switch to open and close such that at no time is one of the output lines electrically connected to one of the input lines of opposite polarity.

### DRAWINGS

Embodiments of the present invention are described below with reference to the following drawings, in which:

FIG. 1 shows an implantable medical device comprising a first module and a second module;

FIG. 2 shows an exemplary switching arrangement for the medical device of FIG. 1 for the two-wire interfaces;

FIG. 3A shows one example of an AC voltage waveform generated by the implantable medical device of FIG. 1 having two phases;

FIG. 3B shows another example of an AC voltage waveform generated by the implantable medical device of FIG. 1 having a third phase;

FIG. 4A shows an embodiment of a circuit usable with the implanted medical device of FIG. 1 in which the opening of the switches is controlled according to current level;

FIG. 4B shows the switch control blocks of FIG. 4A in more detail;

FIG. 4C shows the logic circuits of the switch control blocks of FIG. 4A;

FIG. 5A shows a waveform of a signal on the two wires in the arrangement of FIG. 4A;

FIG. 5B shows a waveform of the Φ1 clocking signal in the power source module (PSM) of FIG. 4A;

FIG. 5C shows a waveform of the Φ2 clocking signal in the PSM of FIG. 4A;

FIG. 5D shows the output of the comparator 18 of FIG. 4A;

FIG. 5E—shows the output of the comparator 20 of FIG. 4A;

FIG. 5F shows the gate control voltage driving switch 5 in the main stimulator module (MSM) of FIG. 4A;

FIG. 5G—shows the gate control voltage driving switch 6 in the MSM of FIG. 4A;

FIG. 5H shows an approximation waveform of the current flowing in the Φ1 switches in the MSM of FIG. 4A;

FIG. 5I—shows the output voltage of the VΦ1B comparator 13 in the MSM of FIG. 4A;

FIG. 5J—shows the gate control voltage that drives the Φ2 PMOS switch 7 in the MSM of FIG. 4A;

FIG. 5K—shows the gate control voltage that drives the Φ2 NMOS switch 8 in the MSM of FIG. 4A;

FIG. 5L—shows an approximation of the current flowing in the Φ2 switches in the MSM of FIG. 4A;

FIG. 5M shows the output voltage of the VΦ2B comparator 15 in the MSM of FIG. 4A;

FIG. 5N shows the current in the storage capacitor 9 of the MSM of FIG. 4A;

FIG. 5O shows the voltage across the storage capacitor 9 of the MSM of FIG. 4A;

FIG. 6 shows an alternative arrangement of the circuit of FIG. 4A;

FIG. 7A shows a circuit in which the opening of the switches is controlled according to timing;

FIG. 7B shows the switch control block of FIG. 7A in more detail;

FIG. 7C shows an example logic circuit for the switch control block of FIGS. 7A and 7B;

FIG. 8A shows a waveform of a signal on the two wires in the arrangement of FIGS. 7A;

FIG. 8B shows a waveform of the Φ1 clocking signal in the PSM of FIG. 7A;

FIG. 8C shows a waveform of the Φ2 clocking signal in the PSM of FIG. 7A;

FIG. 8D shows the output of the comparator 18 of FIG. 7A;

FIG. 8E shows the output of the comparator 20 of FIG. 7A;

FIG. 8F shows the gate control voltage driving switch 5 in the MSM of FIG. 7A;

FIG. 8G shows the gate control voltage driving switch 6 in the MSM of FIG. 7A;

FIG. 8H shows the gate control voltage that drives the Φ2 PMOS switch 7 in the MSM of FIG. 7A;

FIG. 8I shows the gate control voltage that drives the Φ2 NMOS switch 8 in the MSM of FIG. 7A;

FIG. 8J shows the current in the storage capacitor 9 of the MSM of FIG. 7A;

FIG. 8K shows the voltage stored on the storage capacitor (9) in the MSM of FIG. 7A;

FIG. 9 shows an arrangement for a two wire interface circuit with four switches;

FIG. 10 shows a two wire interface with one switch;

FIG. 11 shows a two wire interface with two switches and providing voltage doubling;

FIG. 12 shows a two wire interface with two switches with no voltage doubling;

FIG. 13 shows a general arrangement for a two wire interface circuit;

FIG. 14 shows a cochlear implant system including an external component, and an internal component;

FIG. 15 shows a cochlear implant system having a power source module removable or separable from the main stimulator module; and

FIG. 16 shows a perspective view of a cochlear implant with which embodiments of the present invention may be implemented.

### DETAILED DESCRIPTION

Aspects of the present invention are generally directed to the transfer of power between implanted components of a medical implant. In embodiments of the present invention, the power is transferred from an implantable direct current (DC) power source positioned in a first hermetically sealed module to an implantable DC power storage device positioned in a second hermetically sealed module. This power transfer is accomplished by converting the DC power from the power source to AC power, and transmitting the AC power across electrical lines extending between the two hermetically sealed modules. Once received in the second hermetically sealed module, the AC power is converted back to DC power and is used to charge the DC power storage device.

The conversions from DC power to AC power, and from AC power to DC power, are controlled to prevent backflow from the DC power storage device to the DC power source. More particularly, the poles of the DC power storage device are never electrically connected to opposite poles of the DC power source.

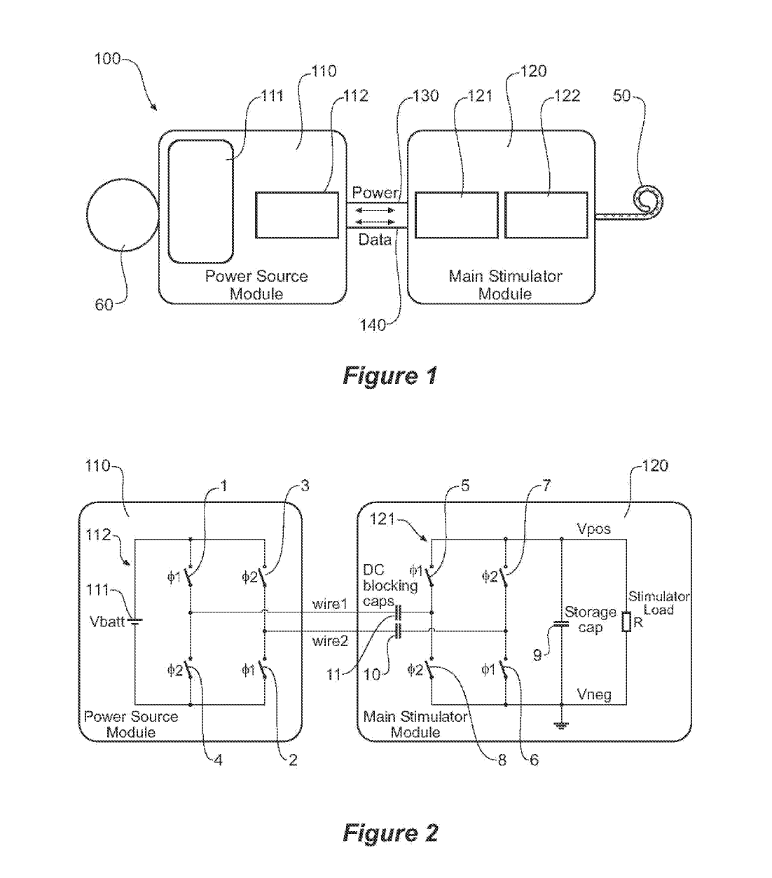

FIG. 1 depicts a medical implant 100 comprising an electrically stimulating medical implant 100. Stimulator 100 may comprise, for example, the implantable part of a medical implant system such as a cochlear implant system. As shown in FIG. 1, medical implant 100, in this example, comprises a first power source module (PSM) 110, and a second a main stimulator module (MSM) 120.

Power source module 110 houses a power source 111 such as a battery, which can be rechargeable and/or removable. If the battery is to be recharged, recharging can be done via coil 60. Coil 60 can also be used for receipt of control signals from the external processor (not shown) as will be understood by the person skilled in the art. Power source module 110 also includes two-wire interface 112 which will be described in more detail below. Two-wire interface 112 interfaces power source 111 with two wires 130 and 140 connecting power source module 110 and main stimulator module 120 as will be described in more detail below.

Main stimulator module 120 includes stimulator control circuitry 122 and another two-wire interface 121, which interfaces between the two wires 130 and 140 and the main stimulator module 120.

Main stimulator module 120 connects to lead 50 supporting an electrode array which in use, is inserted into a recipient's cochlea for normal electrical stimulation as will be understood by the person skilled in the art.

The arrangement shown in FIG. 1 allows for power source module 110 to be removed or otherwise separated from main stimulator module 120. One of the advantages of this is that if power source 111 needs to be replaced or otherwise removed, the surgery required is less serious, invasive or extensive than if the entire medical implant 100 had to be removed. This can also result in minimising loss of the patient's residual hearing.

Any suitable connector can be used to connect power source module 110 to main stimulator module 120. For example, a connector currently used in pacemaker devices can be used.

In the arrangement shown in FIG. 1, two wires 130 and 140 are used to deliver power from power source module 110 to main stimulator module 120. In some instances, two wires 130 and 140 can also be used to transfer data between the two modules.

It will also be appreciated that the direction of power and data flow can be both from the first module or power source module 110 to the second module or main stimulator module 120, or from the second module or main stimulator module 120 to the first module or power source module 110, or a combination of both.

It will also be appreciated that the various aspects described herein need not be applied to a power source module and a main stimulator module of a stimulator, but can instead or in addition to this be applied to a power source module and another type of module or device such as a sensor device or other implantable medical device that is required to obtain power from the power source module.

Embodiments of the present invention prevent exposure of tissue of a recipient to DC current. Accordingly, since there may be an irreducible risk of exposing the power that is transferred between the first module 110 and the second module 120 to tissue (for example in the event of damage to insulation of the wires 130 and/or 140), embodiments of the present invention transmit this power between the two modules as an alternating current (AC) signal.

Accordingly, the function of the two-wire interface 112 in the power source module is to convert the DC power from the power source or battery 111 into an AC signal for transmission on the two wires 130 and 140. In the event that power is being transferred to the power source module 110 from the main stimulator module 120, two-wire interface 112 will act as a rectifying circuit to convert the incoming AC signal to DC to charge the power source 111.

Similarly, the two-wire interface 121 of the main stimulator module 120, will act to convert input AC power transmitted over the two wires 130 and 140 into a DC signal for use in powering circuitry in or associated with main stimulator module 120. Conversely, in the event that power is being transferred from the main stimulator module 120 to power source module 110, two-wire interface 121 will act to convert DC power to AC for transmission on two wires 130 and 140.

In the following section of this description, the examples given will be in relation to power source module 110 supplying power to main stimulator module 120. It will be assumed that power source module 110 will act as the master module and the main stimulator module 120 will act as the slave module, although this need not necessarily always be the case. In some cases, main stimulator module 120 can act as the master and power source module 110 can act as the slave module, irrespective of the direction of flow of power. It will be understood that when it is recited herein that the power module is acting as the master, it is meant that the function of some elements (in this particular example, the two-wire interface 112 generating the AC signal) of the power module act independently of external information, while the main stimulator module, acting as the slave module, will function in accordance with information produced by the master module (in this particular example, the two-wire interface 121 of the main stimulator module 120 will act in accordance with the input AC signal generated by the master module).

Turning now to FIG. 2, there is shown power source module (PSM) 110 and main stimulator module (MSM) 120, having respective two-wire interfaces 112 and 121. As shown, two-wire interfaces 112 and 121 include an arrangement of switches that can be selectively controlled to perform the inverter and rectification functions referred to above.

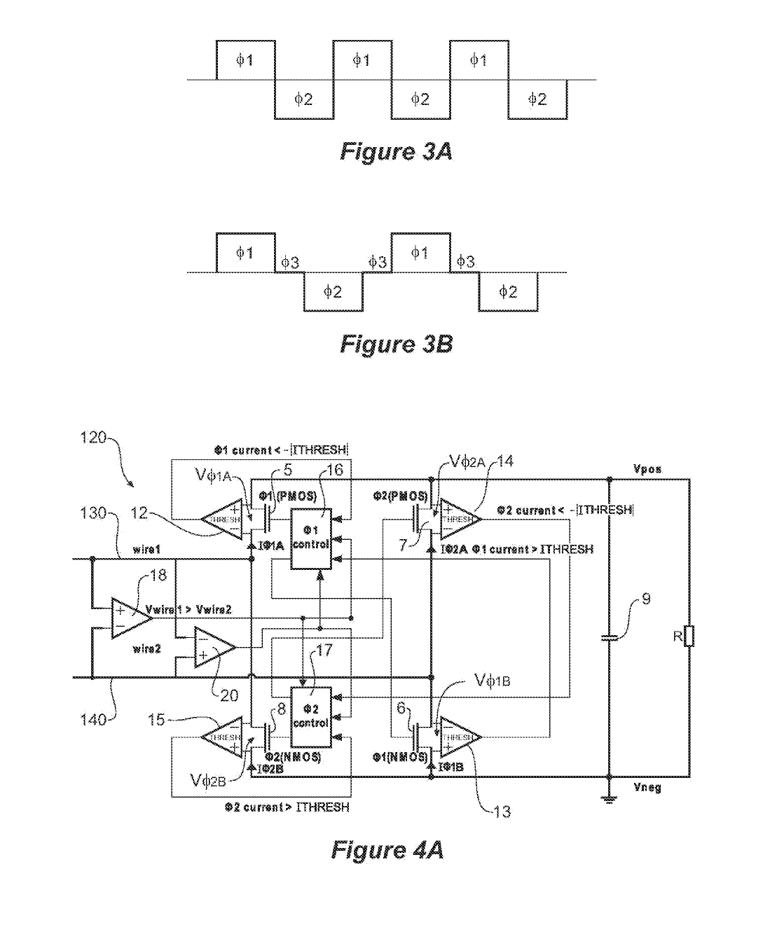

With reference to the PSM (assuming that it is the Master in this example), in this embodiment there are 4 switches, 1, 2, 3 and 4. The switches 1 and 2 labelled Φ1 are closed during a first phase of the AC signal (see FIG. 3A and related description below) and the switches 3 and 4 labelled Φ2 are open during this first phase. This results in a positive voltage difference of about Vbatt between wire 1 and wire 2. Conversely, during a second phase of the AC signal, the Φ2 switches 3 and 4 are closed and the Φ1 switches 1 and 2 are open during the second phase, producing a negative voltage difference of about Vbatt (voltage of the power source or battery 111) between the wire 1 and wire 2. This arrangement accordingly performs an inversion of the DC battery voltage into an AC voltage between the wires, without any DC component.

With reference to the MSM 120 (assuming that it is the slave in this example), four switches 5, 6, 7 and 8 are also provided. The same timing of the Φ1 and Φ2 switches as in the PSM 110 detailed above for the Φ1 and Φ2 switches would rectify the AC voltage between the wires onto a power storage device such as storage capacitor 9 (in this example), with minimal losses compared with a passive diode rectifier.

This arrangement results in current being drawn from the battery in the PSM 110 to charge the storage capacitor 9 in the MSM during both phases.

In one embodiment, use of series capacitors 10 and 11 can be made in the MSM to block any DC current from flowing between either wire and the electrode array connected to the PSM, in the event of a breach of the insulation surrounding the wires. It will however be understood that blocking capacitors 10 and 11 are not necessarily required.

FIG. 3A shows an exemplary signal waveform appearing on wires 130 (wire1) and 140 (wire2). As previously described, the first phase Φ1 is shown as a positive polarity and the second phase Φ2 is shown as a negative polarity in one embodiment. In FIG. 3B there is shown an optional third phase Φ3 which can be provided by timing the switches in the PSM such that there is a period during which they are all open (non-conducting), providing no current on the wires.

In is noted that if the timing of the switching from phase 1 to phase 2 and vice versa is not correct, there will be a time where Vpos can be connected to the negative terminal of the battery 111 or that Vneg will be connected to the positive terminal of battery 111. In this situation, capacitor 9 will actually discharge (i.e., current will backflow from capacitor 9) rather than charge, resulting in a reduction in the efficiency of the power transfer from PSM 110 to MSM 120.

According to one aspect of the present invention herein described, the timing of the switching is sufficiently controlled such that at no time is there provided an electrical path from one polarity of the power storage device 9 or capacitor, to the opposite polarity of the power source or battery 111. This is accomplished in one form by controlling the switches 5, 6, 7 and 8 in the MSM 120, such that a closed (or conducting) switch is caused to be open (non-conducting) prior to a change in polarity of the AC signal. In this way, at no time will there be a return or discharge path to allow the power storage device to discharge into the power source. The opening of a switch is not instantaneous, and that a finite amount of time elapses between the beginning of the opening of the switch to the time that the switch is fully open and non-conducting. During this finite time, the switch is still conducting. Accordingly, beginning to open the switch upon reversal of polarity will result in the switch still momentarily conducting electricity during the reversed polarity, resulting in discharge of the power storage device.

In one embodiment, this is avoided by controlling the switches 5, 6, 7 and 8 to begin opening (or becoming non-conductive) at some time prior to the reversal of polarity of the AC signal that the switch is conducting. In one form, this is accomplished by monitoring a current magnitude in the AC signal and causing the pertinent switch to begin opening when the current magnitude falls below a predetermined threshold.

In another embodiment, the opening of the switches is controlled to occur in accordance with a timing signal rather than with reference to the incoming AC signal.

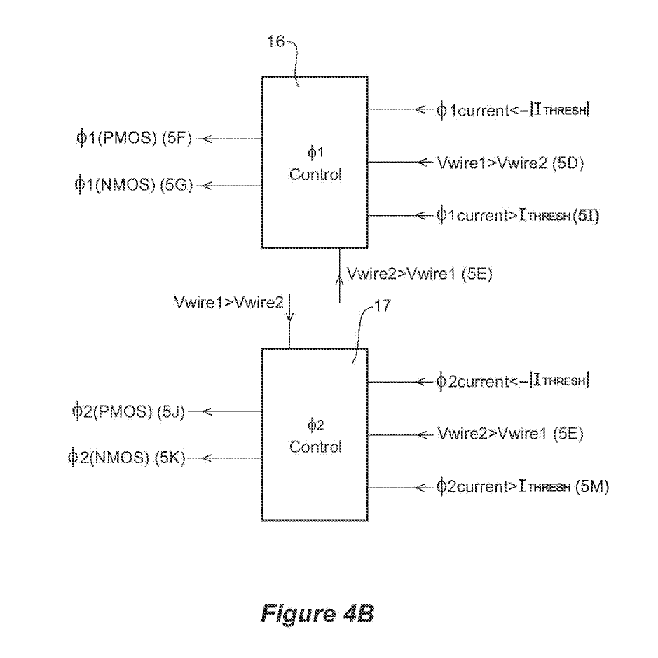

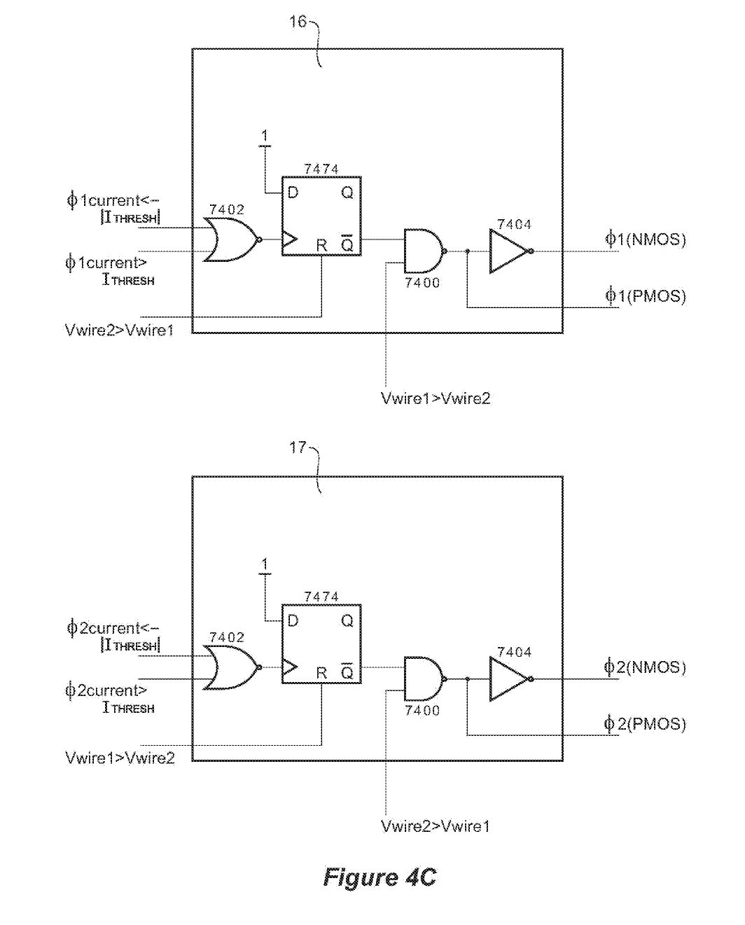

Each of these embodiments will now be described in more detail with reference to FIGS. 4A to 7C.

In FIG. 4A, there is shown one example of a circuit which can be used in the MSM 120. The circuit includes the four switches 5, 6 7 and 8 described above with reference to FIG. 2, respective low-current detectors 12, 13, 14 and 15 to detect when the current through the switches drops below the threshold, switch control blocks 16 and 17, and voltage comparators 18 and 20 for detecting the change in voltage or polarity of the signal on lines 130 and 140. In this example, switches 5, 6, 7 and 8 are provided by MOSFETs.

In the example shown in FIG. 4A, the current detectors are shown as voltage comparators (with high gain) placed across each switch. Because the switches have non-zero resistance, a current through them results in a voltage drop across the switch, so a measurement of the voltage can be considered an equivalent to measuring the current. Once the current has dropped below a threshold (that can be predetermined and set into the comparator), the switches that were closed are opened. Note that four current detectors are shown in this example. However, this number is not necessarily required, and can be determined on a case by case basis per design and required application. For uni-directional power flow (e.g. from left to right, or from PSM 110 to MSM 120) only one current detector is needed for the Φ1 phase and one for the Φ2 phase. It is also possible to provide the current sensing directly in the two input lines 130 and 140 and to control the opening of the switches when it is sensed that the current falls below the threshold.

For example, a current threshold could be selected to be between about 5% and about 50% of the maximum signal value, including about 10%, about 15%, about 20%, about 25%, about 30%, about 35%, about 40%, about 45% and about 50%. In some embodiments, these threshold values can be varied to ‘tune’ the system, depending upon various system parameters such as the level of the signal, the switch resistance and the maximum sensed voltage, as will be understood by the person skilled in the art. In one example, the maximum signal level can be about 100 mA, and the switch resistance can be about 1 Ohm.

One advantage of the arrangement shown in FIG. 4A is that the timing of the switches (for example the frequency and/or duty cycle) in the PSM 110 can change without the MSM 120 having to receive information relating to the state of the PSM 110.

FIG. 4A also shows comparator 20 between wire 1 and wire 2, and also shows current detectors 12, 13, 14 and 15 as having a threshold level prior to triggering. This threshold level can be adjustable in some embodiments.

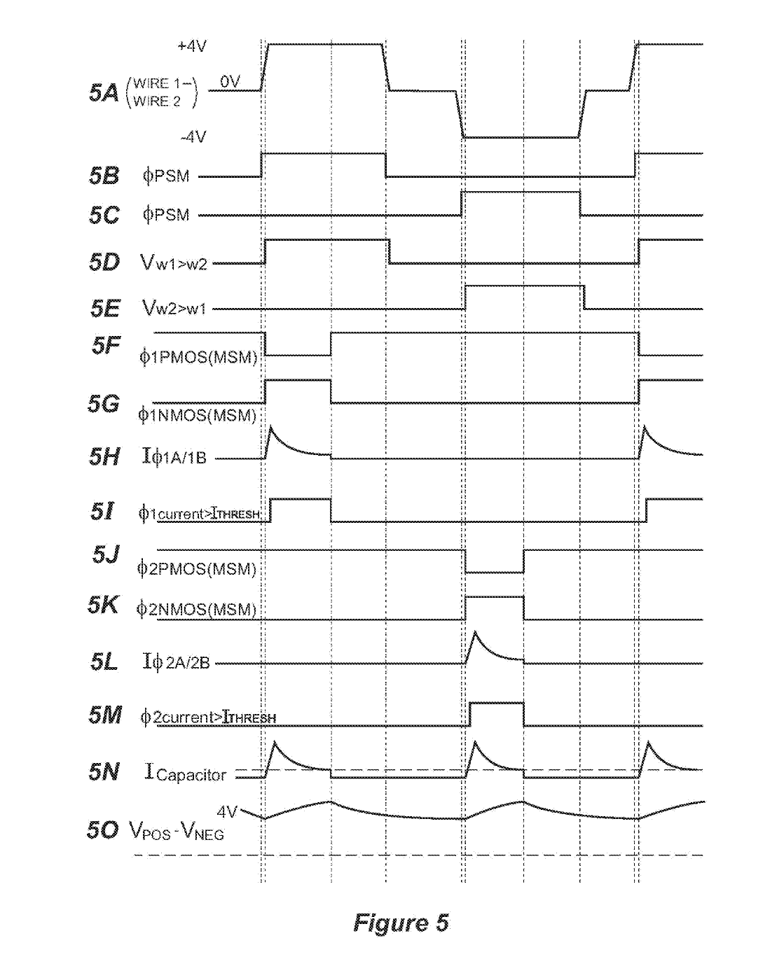

FIG. 4B shows the two switch control blocks 16 and 17 in more detail, showing the pin identifiers and the signal waveforms referred to below with reference to FIGS. 5A to 5O.

FIG. 4C shows the logic circuits for the switch control blocks 16 and 17 of FIGS. 4A and 4B. Example component identifiers for the various logic circuit components are also shown in FIG. 4C. It is noted that this is only one method in which the arrangement can be embodied. In other embodiments, the various logic components can be integrated into a single chip together with the other circuit components (e.g. the switches).

FIGS. 5A to 5O show various waveforms at different points in the circuit of FIGS. 4A and 4B. As can be seen in the various Figures of FIG. 5, when the Φ1 clock goes high, the voltage between Wire1 and Wire2 increases in a finite time, however, in this arrangement, there are no switches closed in the MSM 120, and so there is no negative current spike in the capacitor current. The voltage on the wires goes positive, and the Wire1>Wire2 voltage comparator 18 output goes high, which causes the switch control block 16 to turn on the Φ1 switches 5, 6 in the MSM 120. When these switches turn on, the current through the Φ1 switches 5, 6 spikes up from zero, and gradually decays (note that the capacitor current is the same). When the current decays below the designated threshold, the voltage comparator 13 on the lower Φ1 switch (Φ1B) goes low (this went high when the current first started flowing, but that edge is not used by the switch control block in this particular application). On this edge, the switch control block 16 will turn off the Φ1 switches 5 and 6. Note that the Φ1 clock in the PSM 110 is still high. When that clock goes low, the Φ1 switches in the PSM 110 are opened, and the voltage on the switches between the two wires is no longer driven by the power source, so it is free to return to zero if any load is present to do so.

FIG. 5A shows the voltage difference between the two wires (130 and 140) as driven by the PSM, with finite rise and fall times.

FIGS. 5B and 5C show the Φ1 and Φ2 clocking signals in the PSM (when acting as master). There is a non-overlapping period.

FIG. 5D shows the output of the comparator (18), which goes high when the wire1 (130) voltage is above the wire2 (140) voltage. The output goes low when the wire1 voltage is below the wire2 voltage.

FIG. 5E shows the output of the comparator (20), which goes high when the wire2 (140) voltage is above the wire1 (130) voltage. The output goes low when the wire2 voltage is below the wire1 voltage.

FIG. 5F shows the gate control voltage that drives the Φ1 PMOS switch (5) in the MSM. When the gate is high, the switch is non-conductive; when it is low, the switch is conductive. This signal is driven by the control logic (16) to go low on the rising edge of the signal in FIG. 5D, and is driven by the control logic to go high on the falling edge of the signal in FIG. 5I.

FIG. 5G shows the gate control voltage that drives the Φ1 NMOS switch (6) in the MSM. When the gate is high, the switch is conductive; when it is low, the switch is non-conductive. This signal is driven by the control logic (16) to go high on the rising edge of the signal in FIG. 5D, and is driven by the controller to go low on the falling edge of the signal in FIG. 5I.

FIG. 5H shows an approximation of the current flowing in the Φ1 switches in the MSM. The case shown is when power is flowing in the direction from PSM to MSM. During the period when the Φ1 switches are closed (i.e. as determined by the signals in FIGS. 5F and 5G), there is a positive current flowing from the PSM to MSM. There is an initial spike that decays away towards zero. This drops to zero when the Φ1 switches are opened.

FIG. 5I shows the output voltage of the VΦ1B comparator (13), which effectively amounts to a signal used by the control logic (16) to determine when the Φ1 current is greater than a pre-determined threshold. It is the falling edge of this signal that the control logic will use to determine when to open the Φ1 switches. (Note that the opposite version of this signal, i.e. the output from comparator 12 would be used in the case that power flows from MSM to PSM).

FIG. 5J shows the gate control voltage that drives the Φ2 PMOS switch (7) in the MSM. When the gate is high, the switch is non-conductive; when it is low, the switch is conductive. This signal is driven by the control logic (17) to go low on the rising edge of the signal in FIG. 5E, and is driven by the control logic to go high on the falling edge of the signal in FIG. 5M.

FIG. 5K shows the gate control voltage that drives the Φ2 NMOS switch (8) in the MSM. When the gate is high, the switch is conductive; when it is low, the switch is non-conductive. This signal is driven by the control logic (17) to go high on the rising edge of the signal in FIG. 5E, and is driven by the control logic to go low on the falling edge of the signal in FIG. 5M.

FIG. 5L shows an approximation of the current flowing in the Φ2 switches in the MSM. The case shown is when power is flowing in the direction from PSM to MSM. During the period when the Φ2 switches are closed (i.e. as determined by the signals in FIG. 5J and 5K), there is a positive current flowing from the PSM to MSM. There is an initial spike that decays away towards zero. This drops to zero when the Φ2 switches are opened.

FIG. 5M shows the output voltage of the VΦ2B comparator (15), which effectively amounts to a signal used by the control logic (17) to determine when the Φ2 current is greater than a pre-determined threshold. It is the falling edge of this signal that the control logic will use to determine when to open the Φ2 switches. (Note that the opposite version of this signal, i.e. the output from comparator 14 would be used in the case that power flows from MSM to PSM).

FIG. 5N shows the current in the storage capacitor (9) of the MSM. This amounts to a summation of the two current waveforms in FIG. 5H and 5L. A positive current during Φ1 or Φ2 charges up the capacitor and contributes positively to the efficiency of power transfer over the two-wire link, whereas a negative current during Φ1 or Φ2 discharges the capacitor and contributes negatively to the efficiency. Note that due to the control of the switches in the MSM to open before a change of polarity of the wire1−wire2 voltage as described above, there are no negative current spikes.

FIG. 5O shows the voltage stored on the storage capacitor (9) in the MSM. The voltage charges up during the periods that the Φ1 or Φ2 switches are conducting, and discharges the rest of the time due to the load current drawn from the rest of the circuitry in the MSM (represented by load R). Without the arrangement herein described, there would also be a slight negative transient whenever the polarity of voltage between the wires changes (reflecting a reduced efficiency).

In some embodiments, the same process repeats itself in the reverse direction when the Φ2 clock in the PSM goes high causing its Φ2 switches to close. The wires rapidly reverse polarity, because there are no switches in the MSM closed at this time, and no reverse capacitor current flows.

As can be seen in the capacitor current waveform, I_{CAPACITOR} in FIG. 5N, the current is always positive while the switches are conducting, otherwise it is negative when the switches are not conducting, which means that the voltage across the capacitor keeps building when the capacitor current is non-zero, up to a maximum constant voltage. The voltage reduces as load current discharges it, but is constantly replenished by the charging function described above. At no time does the capacitor 9 discharge back to the PSM as in prior art arrangements, thus providing a highly-efficient power charging circuit.

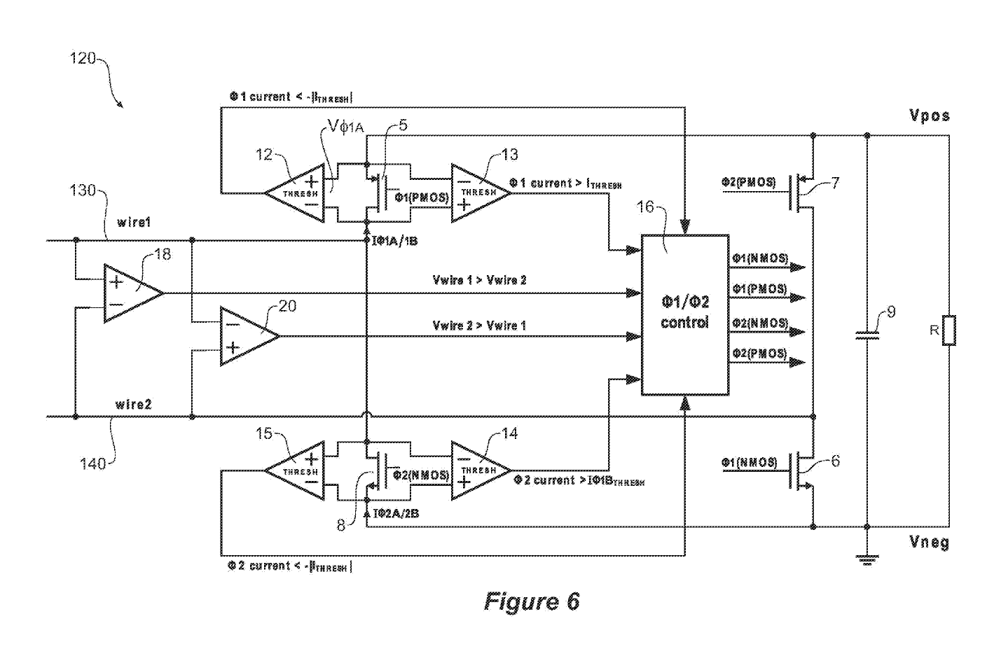

FIG. 6 shows an alternative arrangement of the circuit of FIG. 4A. In this arrangement, current detectors 12, 13, 14 and 15 are all associated with switches 5 and 8. That is, the current detectors are only associated with one of the switches for each phase Φ1 and Φ2, rather than with each of the four switches, as in FIGS. 4A and 4B. In this arrangement, the switch control blocks 16 and 17 of FIG. 4A have also been consolidated into a single switch control block 16 to provide all control functions, although they could alternatively also be provided as two separate switch control blocks 16 and 17.

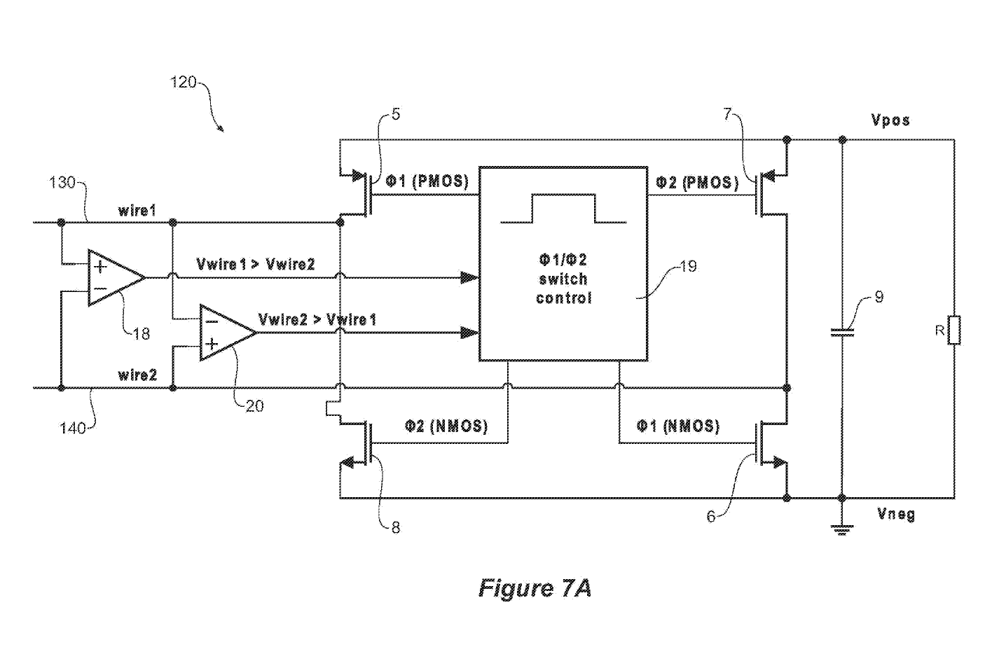

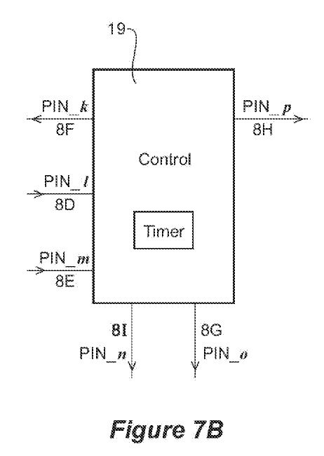

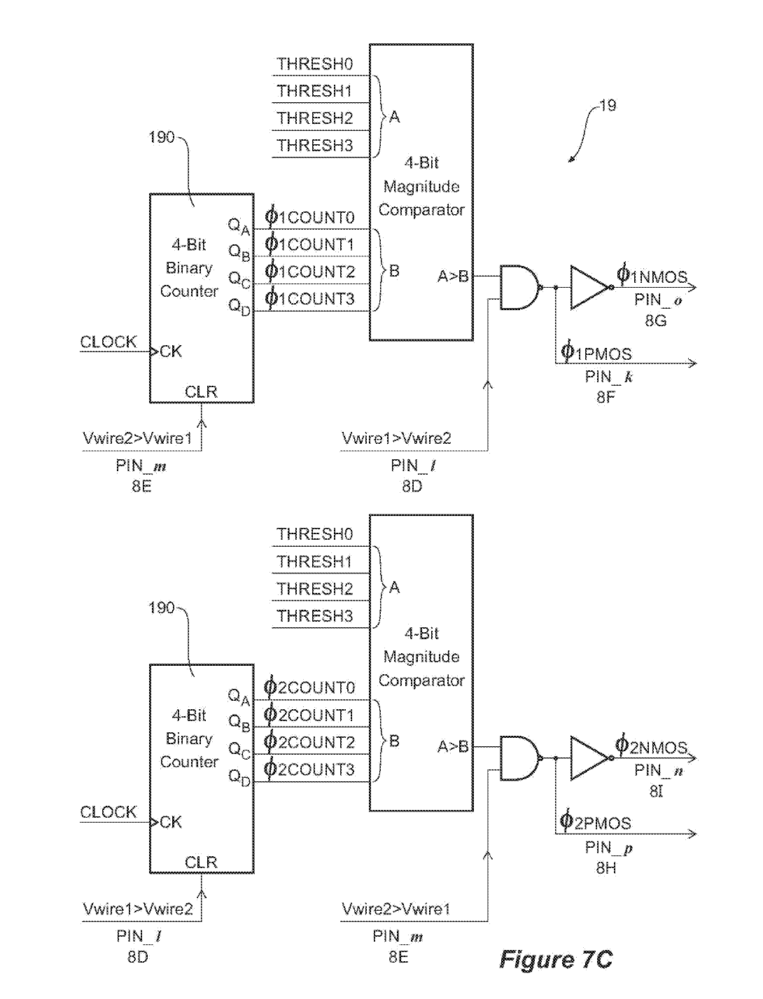

In another embodiment, the switching of the switches in the MSM 120 is controlled by a clock, synchronised with the switches in the PSM 110. FIG. 7A shows a circuit block diagram of a circuit according to this embodiment. Shown are switches 5, 6, 7 and 8, voltage comparator 18 between lines 130 and 140, and storage capacitor 9, as previously described with reference to FIG. 4A. It is noted that in this embodiment, no current detectors for the switches are utilized. Instead of using low-current detectors to determine when to open the switches, a timeout period is employed. For example, when the wire voltage changes from negative to positive, the Φ1 switches will be closed in the MSM in response to this, as before. However, instead of using either the voltage, or a low-current detector, the switches automatically open after a predetermined time. This period should be shorter than the time taken for the voltage on the wires to change in polarity again, hence the efficiency is maximised. This predetermined time can be determined at the time of manufacture of the device and pre-set, or can be determined by an adaptive algorithm as the device operates and can even be communicated via a data link between the PSM and the MSM.

The clocking of the switches in the PSM has not changed (still an independent clock), while the clocking of the switches in MSM 120 is different from the previous method described with reference to FIGS. 4A, 4B, 4C and 5. In this arrangement, when the voltage difference between wire1 and wire2 goes positive, the D1 clock in MSM 120 is caused to go high (for an NMOS switch) (low for the PMOS switch). This stays high for a pre-determined timeout period and then goes low again (or high for the PMOS switch). The timeout period can be generated either by a one-shot circuit or a counter driven by a higher frequency clock. This can be provided as a separate circuit or can be provided as part of switch control 19. It will be noted that this period is designed to be shorter than the Φ1 period in the PSM 110. This can be a fixed value that takes into account the error of the timeout period with respect to that in the PSM. For example, if the PSM Φ1 period is 10 us and the relative error between this and the one-shot circuit in MSM is 10%, then a Φ1 timeout of Bus in PSM 110 could be chosen for correct operation.

The same occurs for Φ2. The result is that there is no negative (discharging) current flowing in the capacitor 9, thereby avoiding the reduction in efficiency that would otherwise occur.

FIG. 7B shows a more detailed view of switch control 19, in the example that the timer is incorporated within. The logic control carried out by switch control 19 will be described in more detail below, after describing the various waveforms of the circuit of FIG. 7A.

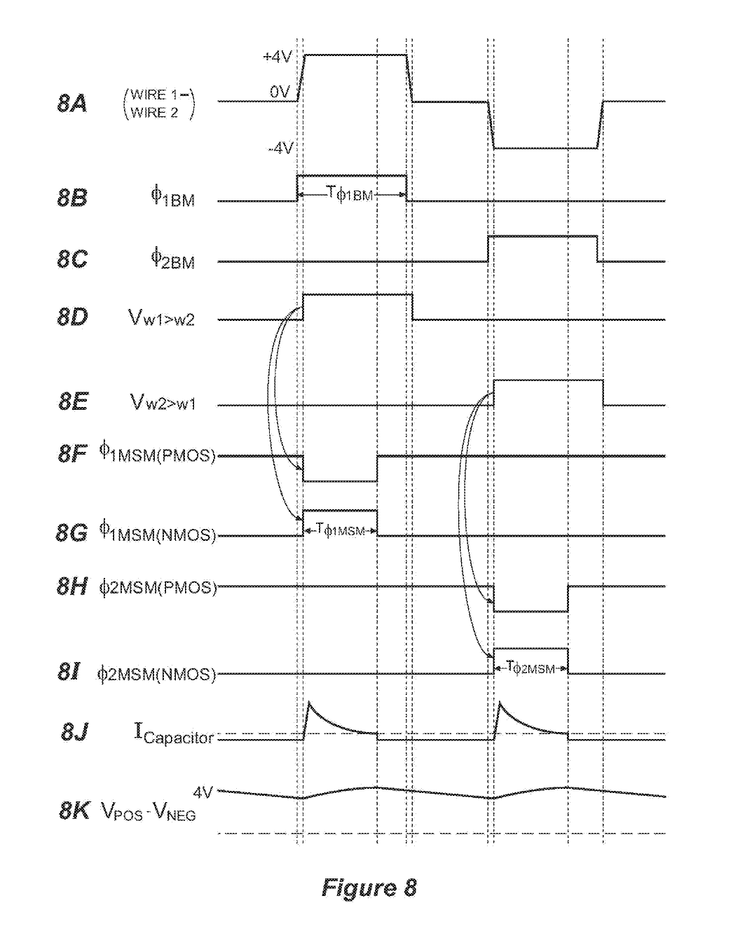

FIGS. 8A to 8K illustrate various waveforms at the points described above in relation to FIG. 7A. Again, as with FIG. 5N, it can be seen that the current I_{CAPACITOR} (FIG. 8I) in the capacitor 9 is never negative while the switches are conducting, with the voltage across the capacitor 9 V_{POS}-V_{NEG} (FIG. 8K) maintaining a relatively constant value of about 4V with a slight ripple imposed over it as the current is used by the load and replenished as described above.

FIG. 8A shows the voltage difference between the two wires (130 and 140) as driven by the PSM, with finite rise and fall times.

FIGS. 8B and 8C show the Φ1 and Φ2 clocking signals in the PSM (when acting as master). There is a non-overlapping period in this example.

FIG. 8D shows the output of the comparator (18), which goes high when the wire1 (130) voltage is above the wire2 (140) voltage. The output goes low when the wire1 voltage is below the wire2 voltage.

FIG. 8E shows the output of the comparator (20), which goes high when the wire2 (140) voltage is above the wire1 (130) voltage. It goes low when the wire2 voltage is below the wire1 voltage.

FIG. 8F and 8G are used for the same purpose as those described in FIG. 5F and 5G respectively, however their means of generation is different. The rising edge of the gate control for Φ1 NMOS switch (falling edge for gate control of Φ1 PMOS switch) is still caused by the control logic (19) to occur on the rising edge of the signal in FIG. 8D. However, the falling edge of the gate control for Φ1 NMOS switch (rising edge for gate control of Φ1 PMOS switch) is caused by a timing mechanism within the control logic (19). For example, when the timer reaches the preset timeout of for example, 8 us.

FIG. 8H and 8I are used for the same purpose as those described in FIG. 5J and 5K respectively, however their means of generation is different. The rising edge of the gate control for Φ2 NMOS switch (falling edge for gate control of Φ2 PMOS switch) is still caused by the control logic (19) to occur on the rising edge of the signal in FIG. 8E. However, the falling edge of the gate control for Φ2 NMOS switch (rising edge for gate control of Φ2 PMOS switch) is caused by a timing mechanism within the control logic (19).

FIG. 8J is the same as FIG. 5N, although the duration is not necessarily identical.

FIG. 8K shows the voltage stored on the storage capacitor (9) in the MSM. The voltage charges up during the periods that the Φ1 or Φ2 switches are conducting, and discharges the rest of the time due to the load current drawn from the rest of the circuitry in the MSM (represented by load R in FIG. 7). Without the arrangement described herein, there would also be a slight negative transient whenever the polarity of voltage between the wires changes (reflecting a lower efficiency in power transfer).

In a further alternative, the circuit can be made programmable and if a data link is available between the PSM module 110 and the MSM module 120, then information about the switching frequency could be sent from PSM 110 to MSM 120 to improve the range of frequencies over which the system could operate.

If the pulse width or switching frequency of the switches in the PSM is variable, this can be addressed by the use of an algorithm in the Φ1/Φ2 switch control block 19 in the MSM 120 to learn what the timeout period or a predetermined time, needs to be, by observing the voltage on the wires over a switching cycle, and adjusting the timeout accordingly.

For example, there could be provided a higher frequency clock in the MSM 120 (at least 10× the switching frequency of the PSM 110) which can be used to count the number of cycles used for the Φ1 and Φ2 pulse widths (via the two voltage comparators 18, 20). For example, if the MSM 120 counts that the Φ1 pulse width is 10 cycles, then in the next cycle it will only keep the Φ1 switches in MSM 120 closed for 9 cycles and then open them. In this way, there is only an error of 10% and will still provide the advantages of the arrangements described even if the PSM 110 changes frequency (or even just duty cycle). The same applies to Φ2.

Referring back to FIG. 7B, there is shown switch control 19 from the circuit of FIG. 7B with pins i to n and associated signals referenced to FIGS. 8A to 8K.

FIG. 7C shows an example logic circuit for the switch control 19 of FIG. 7A and 7B. In this arrangement, an internal CLOCK signal is used to clock a 4-bit binary counter 190. This signal is compared with another internal signal THRESH0, THRESH1, THRESH2, THRESH3 (the “THRESH” bus). This can be hardwired or can be adjusted dynamically by a circuit that tracks the phase width of the signal generated by the master module (for example Power Source Module 110—see FIG. 1).

In the case of hardwiring, and in an example case in which the phase width of the signal generated by the master module is about 10 us, if the CLOCK signal is a 1 MHz digital clock, and THRESH is hardwired to a value of 7, then the circuit will open the relevant phase switches after 7 us when the output of the comparator goes low. This example provides sufficient margin of error between clock frequencies generated in both the master and slave modules.

In another embodiment, the control for switch control block 19 can be programmed in a microcontroller for example. An example of pseudocode for the logic of switch control 19 is as follows:
Set Timeout Period=8 us {Set Timeout Period to 8 us}
Set Timer=0 {Reset timer to 0}
If PIN_l→1, PIN_k=0 {If Vwire1>Vwire2, then turn on switch 5}
If PIN_l→1, PIN_o=1 {If Vwire1>Vwire2, then turn on switch 6}
Start Timer {Start Timer incrementing}
If Timer=Timeout Period then PIN_k=1 {When time since switch 5 on reaches 8 us, turn off switch 5}
If Timer=Timeout Period then PIN_o=0 {When time since switch 6 on reaches 8 us, turn off switch 6}
Set Timer=0 {Reset timer to 0}
If PIN_m→1, PIN_p=0 {If Vwire2>Vwire1 then turn on switch 7}
If PIN_m→0, PIN_n=1 {If Vwire2>Vwire1 then turn on switch 8}
Start Timer {Start Timer incrementing}
If Timer=Timeout Period then PIN_p=1 {When time since switch 7 on reaches 8 us, turn off switch 7}
If Timer=Timeout Period then PIN_n=0 {When time since switch 8 on reaches 8 us, turn off switch 8}

The above has described various embodiments for providing transfer of power from one module to another, using a rectification circuit having high efficiency. It will be understood that many variations and modifications are possible. For example, as previously described, the various aspects described will also work if power flows from the MSM 120 to the PSM 110 (this would imply that a source of power is present in the MSM 120). That is, power can flow bidirectionally in this arrangement. Secondly, it is not necessary that the PSM 110 be the master. That is, the MSM 120 could be the master with autonomous control over its own switches, whilst the PSM 110 could be the slave with the appropriate switch control circuitry as described previously. This distinction can be made regardless of the direction of power flow.

Furthermore, for the current detector method of determining when to open the switches in the slave module, the current detection does not need to be limited to detecting the current directly in one (or more) of the switches. It could detect the current flowing in a device in series with the switches.

Other current detection mechanisms could also be used, instead of the voltage comparators across resistive switches shown in FIG. 4A. Examples include a hall-effect sensor in series with the switches, or an inductive element in series.

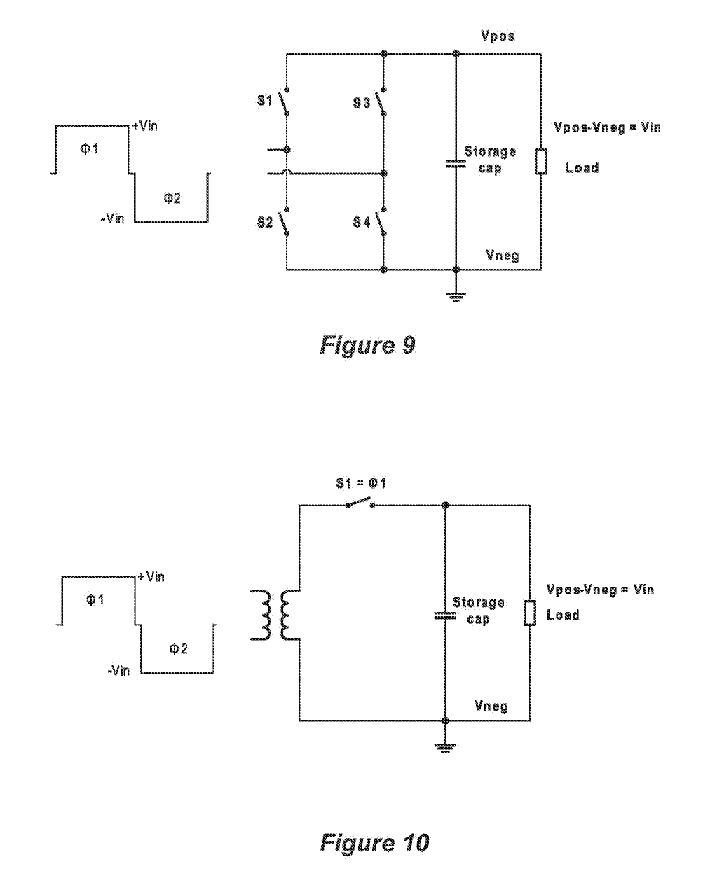

Following, various different embodiments and variations are described, with reference to FIGS. 9 to 12. FIG. 9 shows a generalised slave module (which could be either the PSM 110 or MSM 120) having a four-switch configuration. Also shown in FIG. 9 is a general waveform of the signal on the wires 130, 140, generated by the master module (which could be the other of the MSM 120 or MSM 110). While in previous examples, it has been shown that this waveform can be generated by a similar four-switch arrangement in the master module, as shown in FIG. 2, it will be appreciated that in some embodiments, this waveform can be generated by any suitable means including a standard waveform generator, whether by analogue circuit arrangement and/or digital circuit arrangement.

As previously described, in the four-switch arrangement, switches S1 and S4 are closed during Φ1, and switches S2 and S3 are closed during Φ2. This configuration can be used in certain arrangements such as a capacitively coupled link (as in the arrangement of FIG. 2), or a transformer coupled link.

FIG. 10 shows an arrangement with a single switch S1. This arrangement is equivalent to the general arrangement of FIG. 9, with switch S1 closed during Φ1, switches S2 and S3 permanently open, or non-existent, and switch S4 permanently closed, or replaced by a short-circuit.

This arrangement can be used in a transformer or direct-coupled link (i.e. with no transformer), and acts as a half-wave rectifier, except that current can flow in both directions.

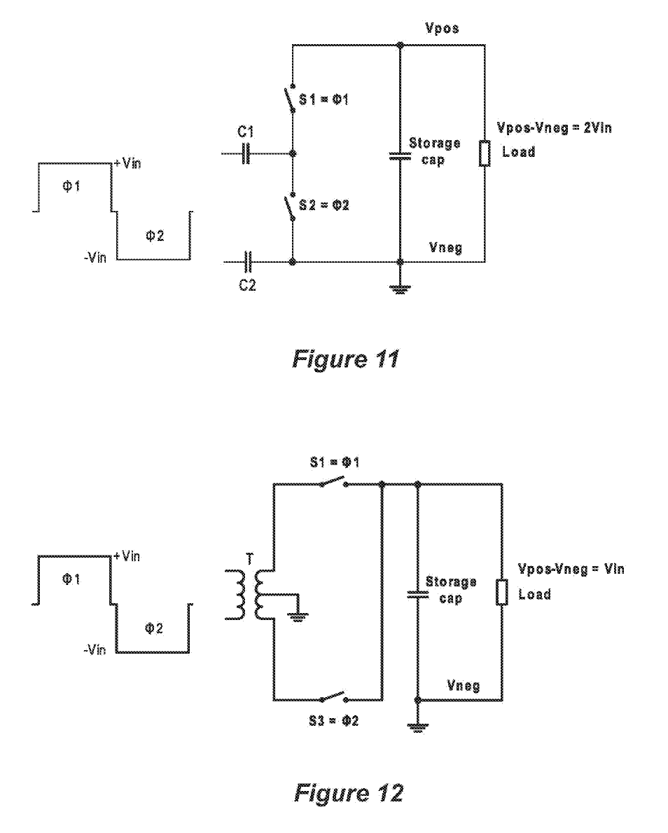

FIG. 11 shows a two-switch topology, which also acts as a voltage doubler, with capacitors C1 and C2 involved in the voltage doubling. In this arrangement, switch S1 is closed during Φ1, switch S2 is closed during Φ2, switch S3 is permanently open or is non-existent and switch S4 is permanently closed or replaced by a short circuit.

In operation, each capacitor C1 and C2 charges up during Φ2, and then this voltage is added to the source voltage during Φ1, providing twice the voltage of the source voltage on the storage device or capacitor 9 (in this example). For example, if the source voltage is about 4V, the voltage across storage capacitor 9 will be about 8V. It will be appreciated that other arrangements providing further voltage multiplication (e.g. tripler or quadrupler) are also possible.

FIG. 12 also shows a two-switch arrangement, providing a one-to-one voltage transfer. This arrangement is equivalent to a full-wave rectifier, except that current can flow in both directions. In this arrangement, switch S1 is closed during Φ1, switch S3 is closed during Φ2, and switches S2 and S4 are permanently open or non-existent.

In this arrangement, centre-tapped transformer T centres the signal coming in from the two wires 130 and 140, on Vneg. Only two switches are used to conduct current in each phase between both modules. Alternatively, a non-centre tapped transformer arrangement could use all four switches, providing an arrangement equivalent to that of FIG. 9.

It will be appreciated that these various arrangements can be provided by either providing four switches and providing appropriate control signals to keep them closed or open as required to provide the required number of switches, or can be provided by manufacturing the circuit with the required number of switches and open/closed circuits.

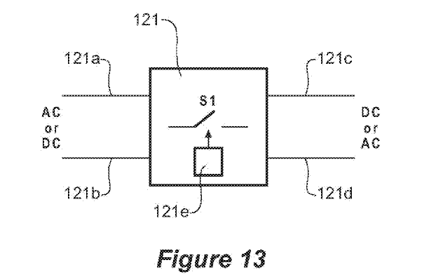

In a broad form then, there has been disclosed a two wire interface circuit for converting between an alternating current (AC) signal and a direct current (DC) signal, as shown in FIG. 13. FIG. 13 shows the two wire interface circuit 121 two input lines, referred to as interface connection lines 121a, 121b, two output lines, referred to as storage connection lines 121c, 121d; at least one switch S1 disposed between the input interface connection lines and the output storage connection lines and at least one switch controller 121e for controlling the at least one switch such that at no time is one of the output lines electrically connected to one of the input lines of opposite polarity.

It will be appreciated that in some applications, the input interface connection lines 121a and 121b will carry an AC signal and the output storage connection lines 121c and 121d will carry the DC signal. For example, with reference to FIG. 2, if wire1 (130) and wire2 (140) are carrying the AC signal from the PSM 110, and the two wire interface circuit 121 is in MSM 120, wires 130, 140 will be the input interface connection lines 121a and 121b and the output storage connection lines 121c, 121d will carry the converted DC signal to power storage device or capacitor 9 in the MSM.

In another example, if the power storage device or capacitor 9 is providing a charge for the power source 111 in the PSM 110, the input lines 121a and 121b will be connected to the power storage device or capacitor 9 in the MSM 120, and the input to the two wire interface circuit 121 will be a DC signal from the power storage device 9. The output storage connection lines 121c and 121d in this case will then carry the converted AC signal to deliver the power to the PSM 110. The equivalent two wire interface circuit in the PSM 110 (labelled as 112 in FIG. 2) will act to convert the incoming AC signal to a DC signal to provide the power to the power source 111.

As previously described, switch controller 121e will control switch S1 such that at no time is one of the output lines electrically connected to one of the input lines of opposite polarity. This thereby prevents the power storage device or capacitor 9 in one example, that is being charged, from discharging temporarily in each cycle, providing a more efficient power transfer.

In one aspect, this is achieved by ensuring that the at least one switch S1 is open (and thus not conducting) prior to the AC voltage signal changing polarity. In one specific embodiment of this aspect, this is achieved by controlling the switch S1 such that it begins to open when the AC current signal reduces below a threshold.

The timing can be provided by a measurement on the AC waveform made at the two wire interface circuit, a measurement of timing made from the closing of the switch S1, or from timing or other data transmitted from the master module for example. A specific example of this embodiment is described above in relation to FIGS. 7A and 7B and 8.

It will be appreciated that the number of switches in two wire interface circuit 121 can be selected as required, and can include 1, 2, 3, 4, or more as previously described.

Other variations and additions are also possible, such as those described in a co-pending application entitled “Power Transfer in the Presence of Tissue”, previously incorporated by reference.

In another aspect, there is also provided a method for converting between an alternating current (AC) signal and a direct current (DC) signal. The method involves causing the at least one switch to open and close such that at no time is one of the output lines electrically connected to one of the input lines of opposite polarity.

In one example, when the at least one switch is closed and is caused to open, the method results in opening the at least one switch prior to a change in polarity of the input AC signal such that the at least one switch is completely open when the polarity changes. As previously described, this can be accomplished in a number of ways. For example, in one way, it can be done by opening the at least one switch upon a current through the at least one switch reducing to or below a threshold. In another example, this can be done by opening the at least one switch a predetermined time after closing.

As described above and with reference to FIGS. 4A, 4B, 7A and 7B for example, there is also provided a switch controller for controlling at least one switch in a circuit for converting between an alternating current (AC) signal and a direct current (DC) signal. The circuit comprises two input lines, two output lines and the least one switch disposed between the input lines and the output lines. The switch controller is configured to control the at least one switch to open and close such that at no time is one of the output lines electrically connected to one of the input lines of opposite polarity.

In one example, the switch controller is configured so that when the at least one switch is closed and is caused to open, the at least one switch is caused to open prior to a change in polarity of the input AC signal such that the at least one switch is completely open when the polarity changes.

Again as previously described, this can be accomplished in a number of ways, including in one way, configuring the controller to control the at least one switch so that the at least one switch is caused to open upon a current through the at least one switch reducing to or below a threshold. In another example, the at least switch one is caused to open a predetermined time after closing.

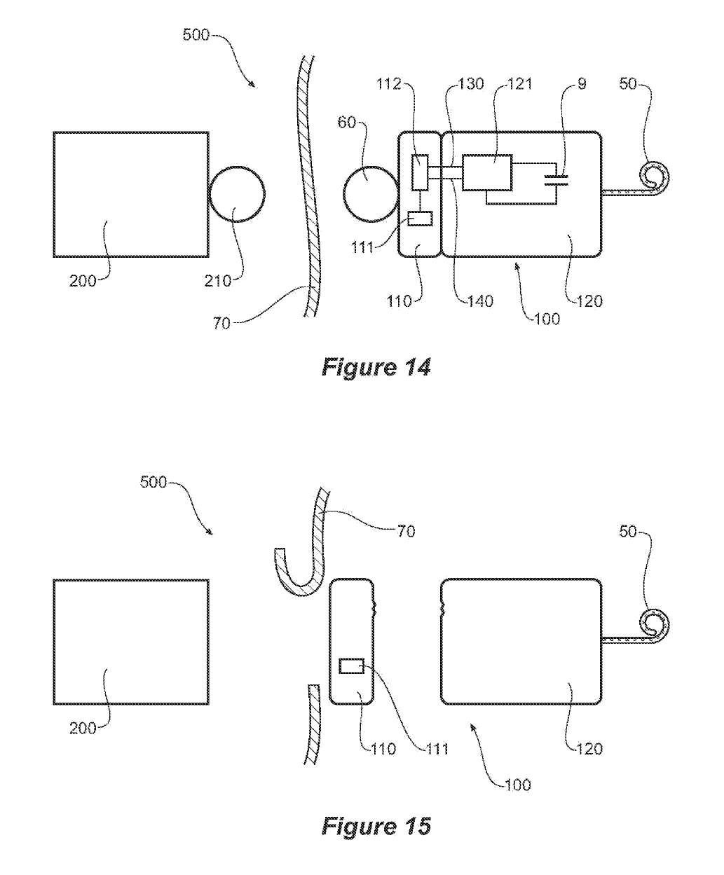

FIG. 14 shows a medical implant system, in this case a cochlear implant system 500, having an external component 200, being a sound processor, and an internal component 100 or medical implant, in this case, being a stimulator, implanted into an implantee (not shown), under tissue 70.

In operation, processor 200 receives input signals in the form of sound information from the surrounding area around the implantee via any suitable means, such as a microphone (not shown) and processes this data into control signals for transmission to the internal component or stimulator 100. The control signals are transmitted transcutaneously across tissue 70 via transmitting coil 210, to be received by receiving coil 60 of the internal component. The control signals are then further processed by the circuitry in the medical implant 100 to provide stimulation signals for applying directly to the cochlea of the implantee via electrode array 50 as will be understood by the person skilled in the art.

The circuitry in the stimulator is in this application, powered by the voltage developed across power storage device 9 (such as a capacitor) as previously described. This power storage device or capacitor 9 is charged by power source or battery 111 in power source module (PSM) 110, via circuitry 112 in the power source module (PSM) 110 and circuit 121 in the internal component or, in this case, main stimulator module (MSM) 120 and wires 130 and 140 as previously described. In some embodiments, the transmitted signal from the external component to the internal component or implant can also contain a power element which is extracted in the power source module 110 to recharge power source or battery 111.

In some embodiments, as shown in FIG. 14, first module or power source module 110 can be removed or separated from second module or main stimulator module 120 to enable replacement of battery 111 and then reconnected to main stimulator module 120. This enables a much simpler and less evasive procedure for changing the battery or for performing other maintenance to the power source module. In this aspect or embodiment, the circuit 112 and circuit 121 can be present, but can also be omitted. It will also be appreciated that receive coil 60 can be connected to power source module 110 or to main stimulator module 120.

In a broad form then, there is provided a medical implant 100 comprising a first module 110 comprising a DC power source 111 and a DC/AC converter 112 for converting between a DC signal and an AC signal. The medical implant 100 also comprises a second module 120 comprising functional electronics, a DC power storage device 9 for providing power to the functional electronics; and a circuit 121 for converting between an AC signal and a DC signal. In one form, this circuit comprises two lines 130, 140 electrically connected to the DC/AC converter 112, two lines electrically connected to the DC power storage device 9, at least one switch disposed between the two lines electrically connected to the DC/AC converter and the two lines electrically connected to the DC power storage device; and at least one switch controller for controlling the at least one switch such that at no time is the DC power storage device electrically connected to an opposite polarity of the DC power source.

In a broad form, there is also provided a medical implant system 500 comprising an external component 200 for receiving input signals and converting the received input signals into control signals, and an internal component 100 for receiving the control signals. As described previously, the internal component comprises a first module 110 comprising a DC power source 111 and a DC/AC converter 112 for converting between a DC signal and an AC signal. The medical implant 100 also comprises a second module 120 comprising functional electronics, a DC power storage device 9 for providing power to the functional electronics; and a circuit 121 for converting between an AC signal and a DC signal. In one form, this circuit comprises two lines 130, 140 electrically connected to the DC/AC converter 112, two lines electrically connected to the DC power storage device 9, at least one switch disposed between the two lines electrically connected to the DC/AC converter and the two lines electrically connected to the DC power storage device; and at least one switch controller for controlling the at least one switch such that at no time is the DC power storage device electrically connected to an opposite polarity of the DC power source.

FIG. 15 shows a removal procedure in which power source module 110 has been separated from main stimulator module 120 and is being removed via an incision in tissue 70 of the implantee.

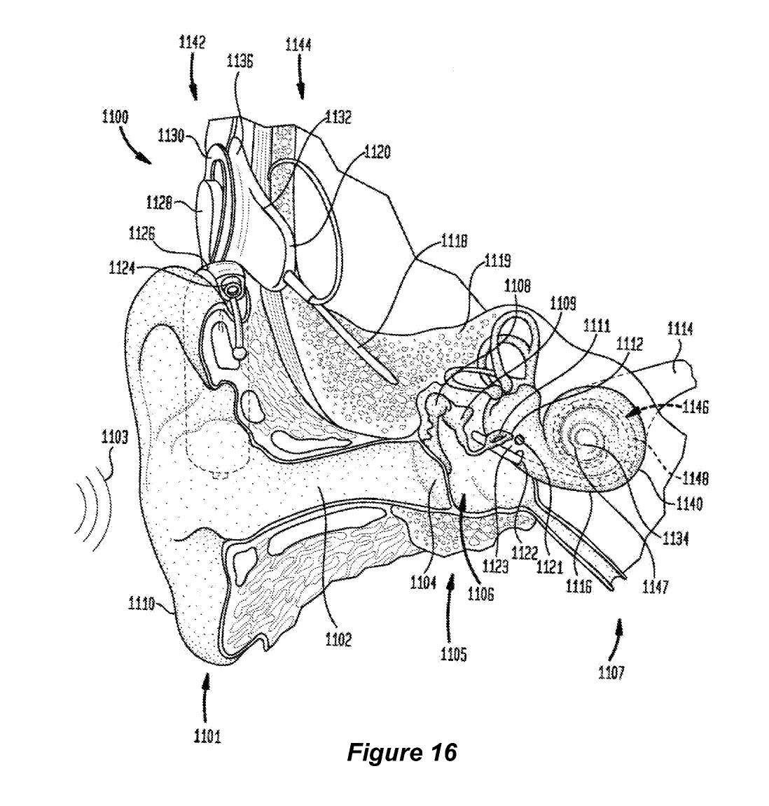

FIG. 16 is perspective view of a cochlear implant, referred to as cochlear implant 1100, implanted in a recipient. The recipient has an outer ear 1101, a middle ear 1105 and an inner ear 1107. Components of outer ear 1101, middle ear 1105 and inner ear 1107 are described below, followed by a description of cochlear implant 1100.

In a fully functional ear, outer ear 1101 comprises an auricle 1110 and an ear canal 1102. An acoustic pressure or sound wave 1103 is collected by auricle 1110 and channeled into and through ear canal 1102. Disposed across the distal end of ear cannel 1102 is a tympanic membrane 1104 which vibrates in response to sound wave 1103. This vibration is coupled to oval window or fenestra ovalis 1112 through three bones of middle ear 1105, collectively referred to as the ossicles 1106 and comprising the malleus 1108, the incus 1109 and the stapes 1111. Bones 1108, 1109 and 1111 of middle ear 1105 serve to filter and amplify sound wave 1103, causing oval window 1112 to articulate, or vibrate in response to vibration of tympanic membrane 1104. This vibration sets up waves of fluid motion of the perilymph within cochlea 1140. Such fluid motion, in turn, activates tiny hair cells (not shown) inside of cochlea 1140. Activation of the hair cells causes appropriate nerve impulses to be generated and transferred through the spiral ganglion cells (not shown) and auditory nerve 1114 to the brain (also not shown) where they are perceived as sound.

Cochlear implant 1100 comprises an external component 1142 (which, in some embodiments, corresponds to external component 200 detailed above with respect to FIGS. 14 and 15) which is directly or indirectly attached to the body of the recipient, and an internal component 1144 (which, in some embodiments, corresponds to the internal component 100 detailed above with respect to FIGS. 14 and 15) which is temporarily or permanently implanted in the recipient. External component 1142 typically comprises one or more sound input elements, such as microphone 1124 for detecting sound, a sound processor 1126, a power source, such as a battery for example (not shown), and an external transmitter unit 1128. External transmitter unit 1128 comprises an external coil 1130 and, preferably, a magnet (not shown) secured directly or indirectly to external coil 1130. Sound processor 1126 processes the output of microphone 1124 that is positioned, in the depicted embodiment, by auricle 1110 of the recipient. Sound processor 1126 generates encoded signals, sometimes referred to herein as encoded data signals, which are provided to external transmitter unit 1128 via a cable (not shown). Sound processor 1126 may further comprise a data input interface (not shown) that may be used to connect sound processor 1126 to a data source, such as a personal computer or musical instrument (e.g., a MIDI instrument).

Internal component 1144 comprises an internal receiver unit 1132, a stimulator unit 1120, and a stimulating lead assembly 1118. Internal receiver unit 1132 comprises an internal coil 1136, and preferably, a magnet (also not shown) fixed relative to the internal coil. Internal receiver unit 1132 and stimulator unit 1120 (which, in some embodiments, corresponds to the stimulator discussed above) are hermetically sealed within separate or shared biocompatible housings, sometimes collectively referred to as a stimulator/receiver unit. In an exemplary embodiment, an implantable power source in a module separate from the stimulator unit (e.g., the internal receiver unit 1132) and/or the stimulator/receiver unit provides power to these units via an electrical connection corresponding to that detailed above. The internal coil receives stimulation data and/or power from external coil 1130. Stimulating lead assembly 1118 has a proximal end connected to stimulator unit 1120, and a distal end implanted in cochlea 1140. Stimulating lead assembly 1118 extends from stimulator unit 1120 to cochlea 1140 through mastoid bone 1119. In some embodiments stimulating lead assembly 1118 may be implanted at least in basal region 1116, and sometimes further. For example, stimulating lead assembly 1118 may extend towards apical end of cochlea 1140, referred to as cochlea apex 1134. In certain circumstances, stimulating lead assembly 1118 may be inserted into cochlea 1140 via a cochleostomy 1122. In other circumstances, a cochleostomy may be formed through round window 1121, oval window 1112, the promontory 1123 or through an apical turn 1147 of cochlea 1140.

Stimulating lead assembly 1118 comprises a longitudinally aligned and distally extending array 1146 of electrode contacts 1148, sometimes referred to as array of electrode contacts 1146 herein. Although array of electrode contacts 1146 may be disposed on stimulating lead assembly 1118, in most practical applications, array of electrode contacts 1146 is integrated into stimulating lead assembly 1118. As such, array of electrode contacts 1146 is referred to herein as being disposed in stimulating lead assembly 1118. Stimulator unit 1120 generates stimulation signals which are applied by electrode contacts 1148 to cochlea 1140, thereby stimulating auditory nerve 1114. Because, in cochlear implant 1100, stimulating lead assembly 1118 provides stimulation, stimulating lead assembly 1118 is sometimes referred to as a stimulating lead assembly.

In cochlear implant 1100, external coil 1130 transmits electrical signals (that is, power and stimulation data) to internal coil 1136 via a radio frequency (RF) link. Internal coil 1136 is typically a wire antenna coil comprised of multiple turns of electrically insulated single-strand or multi-strand platinum or gold wire. The electrical insulation of internal coil 1136 is provided by a flexible silicone molding (not shown). In use, implantable receiver unit 1132 may be positioned in a recess of the temporal bone adjacent auricle 1110 of the recipient.

While the various aspects have been described with specific reference to a cochlear implant, it will be understood that the principles of the various aspects described can be applied to other types of medical implants. For example:

ABI (Auditory Brainstem Implant, electrode for hearing, placed in the brainstem) such as Cochlear Corporation's Nucleus 24 [R] Multichannel Auditory Brainstem Implant (Multichannel ABI)

The auditory brainstem implant consists of a small electrode that is applied to the brainstem where it stimulates acoustic nerves by means of electrical signals. The stimulating electrical signals are provided by a signal processor processing input sounds from a microphone located externally to the user. This allows the user to hear a certain degree of sound.

FES (Functional Electrical Stimulation)

FES is a technique that uses electrical currents to activate muscles and/or nerves, restoring function in people with paralysis-related disabilities.

Injuries to the spinal cord interfere with electrical signals between the brain and the muscles, which can result in paralysis.

SCS (Spinal Cord Stimulator).

This system delivers pulses of electrical energy via an electrode in the spinal area and can be used for pain management. An example of a commercially available system is the RESTOREPRIME system by Medtronic, Inc, USA.

In an exemplary embodiment of the present invention, there is a medical implant, a medical implant system, and a method for transferring power in a medical implant from a first module to a second module via a connection between the modules. A switch controller controls at least one switch so that at no time is a DC power storage device in one module electrically connected to an opposite polarity of a DC power source in the other module, thereby increasing the efficiency of power transfer from the first module to the second module. In one embodiment, the medical implant is a cochlear implant and the medical implant system is a cochlear implant system. According to

According to an exemplary embodiment of the present invention, there is a circuit for converting between an alternating current (AC) signal and a direct current (DC) signal. The circuit comprises two input lines, two output lines and a switch disposed between the input lines and the output lines. A switch controller is also provided for controlling the at least one switch such that at no time is one of the output lines electrically connected to one of the input lines of opposite polarity. In an exemplary embodiment, the switch is caused to open prior to a change in polarity of the input AC signal such that the at least one switch is completely open when the polarity changes. In another exemplary embodiment, the switch is caused to open upon a current through the switch reducing below a threshold. In yet a further form, the switch is caused to open a predetermined time after closing. In some embodiments, there are four switches.

According to another exemplary embodiment of the present invention, a circuit for converting between an alternating current (AC) signal and a direct current (DC) signal is provided. In this exemplary embodiment, a first, second, third and fourth switch are provided, as well as a switch controller to control the switches to close when their respective AC signals change polarity and to open prior to when their respective AC signals change polarity so that the opening switches are completely open when the polarity changes.

In another exemplary embodiment of the present invention, there is a medical implant that comprises a first module and a second module. The first module is provided with a DC power source and a DC/AC converter for converting between a DC signal and an AC signal. The second module comprises functional electronics and a DC power storage device for providing power to the functional electronics. A circuit for converting between an AC signal and a DC signal is also provided in the second module, and the circuit is provided with a switch controller for controlling switches in the circuit so that at no time is the DC power storage device electrically connected to an opposite polarity of the DC power source, thereby increasing the efficiency of power transfer from the first module to the second module. As noted herein, in some embodiments, the medical implant is a cochlear implant.

Another exemplary embodiment of the present invention includes a medical implant system comprising a medical implant as detailed herein and an external component. In one embodiment, the medical implant system is a cochlear implant system, where the medical implant is the implanted portion of the cochlear implant and the external component is a processor of the cochlear implant system.

An exemplary embodiment includes a method for use in a circuit comprising two input lines, two output lines and a switch disposed between the input lines and the output lines. The method causes the switch to open and close so that at no time is one of the output lines electrically connected to one of the input lines of opposite polarity. The method can be applied to a circuit comprising one, two, three, four or more switches.

An exemplary embodiment further includes a switch controller for use in an exemplary circuit as described herein and variations thereof, configured to control one or more switches according to the method described, so that at no time is one of the output lines electrically connected to one of the input lines of opposite polarity.

In another exemplary embodiment, there is a medical implant comprising a first module comprising a DC power source and a DC/AC converter for converting between a DC signal and an AC signal, a second module comprising functional electronics, a DC power storage device for providing power to the functional electronics and a circuit for converting between an AC signal and a DC signal The circuit comprises two lines electrically connected to the DC/AC converter, two lines electrically connected to the DC power storage device, at least one switch disposed between the two lines electrically connected to the DC/AC converter and the two lines electrically connected to the DC power storage device, and at least one switch controller for controlling the at least one switch such that at no time is the DC power storage device electrically connected to an opposite polarity of the DC power source.

In another exemplary embodiment, there is a medical implant as described above and/or below, wherein the circuit comprises four switches disposed between the two lines electrically connected to the DC/AC converter and the two lines electrically connected to the DC power storage device. In another exemplary embodiment, there is a medical implant as described above and/or below, wherein switches that are closed are caused to open prior to a change in polarity of the AC signal such that the switches are completely open when the polarity changes. In another exemplary embodiment, there is a medical implant as described above and/or below, wherein the switches that are closed are caused to open upon a current through the closed switches reducing below a threshold. In another exemplary embodiment, there is a medical implant as described above and/or below, wherein the switches that are closed are caused to open a predetermined time after closing. In another exemplary embodiment, there is a medical implant as described above and/or below, wherein the DC power source is a battery. In another exemplary embodiment, there is a medical implant as described above and/or below, wherein the DC storage device is a capacitor. In another exemplary embodiment, there is a medical implant as described above and/or below, wherein the medical implant is a cochlear implant.

In another exemplary embodiment, there is a medical implant system comprising an external component for receiving input signals and converting the received input signals into control signals and an internal component for receiving the control signals, the internal component comprising a first module comprising a DC power source and a DC/AC converter for converting between a DC signal and an AC signal and a second module comprising functional electronics, a DC power storage device for providing power to the functional electronics and a circuit for converting between an AC signal and a DC signal, the circuit comprising two lines electrically connected to the DC/AC converter, two lines electrically connected to the DC power storage device, at least one switch disposed between the two lines electrically connected to the DC/AC converter and the two lines electrically connected to the DC power storage device, and at least one switch controller for controlling the at least one switch such that at no time is the DC power storage device electrically connected to an opposite polarity of the DC power source.

In another exemplary embodiment, there is a medical implant system as described above and/or below, wherein the medical implant system is a cochlear implant system and the external component is a sound processor and the internal component is a stimulator. In another exemplary embodiment, there is a medical implant as described above and/or below, wherein the circuit comprises four switches disposed between the two lines electrically connected to the DC/AC converter and the two lines electrically connected to the DC power storage device. In another exemplary embodiment, there is a medical implant as described above and/or below, wherein switches that are closed are caused to open prior to a change in polarity of the AC signal such that the switches are completely open when the polarity changes. In another exemplary embodiment, there is a medical implant as described above and/or below, wherein the switches that are closed are caused to open upon a current through the closed switches reducing below a threshold. In another exemplary embodiment, there is a medical implant as described above and/or below, the switches that are closed are caused to open a predetermined time after closing. In another exemplary embodiment, there is a medical implant as described above and/or below, wherein the DC power source is a battery. In another exemplary embodiment, there is a medical implant as described above and/or below, wherein thee DC storage device is a capacitor.

In another embodiment, there is a method practiced in a circuit comprising two input lines, two output lines and at least one switch disposed between the input lines and the output lines, the method being practiced for converting between an alternating current (AC) signal and a direct current (DC) signal, the method comprising causing the at least one switch to open and close such that at no time is one of the output lines electrically connected to one of the input lines of opposite polarity. In another exemplary embodiment of this method, when the at least one switch is closed and is caused to open, the method further comprises opening the at least one switch prior to a change in polarity of the input AC signal such that the at least one switch is completely open when the polarity changes. In another exemplary embodiment of this method the method further comprises opening the at least one switch upon a current through the at least one switch reducing below a threshold. In another exemplary embodiment of this method, the method further comprises opening the at least one switch a predetermined time after closing.

While various embodiments of the present invention have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

The reference to any prior art in this specification is not, and should not be taken as, an acknowledgement of any form of suggestion that such prior art forms part of the common general knowledge.

What is claimed is:

1. A medical implant comprising:
a power source configured to generate a direct current (DC) signal; and
a first interface configured to convert the DC signal to an alternating current (AC) signal;
a DC power storage device;
a second interface configured to convert the AC signal received from the first interface via two interface connection lines to a DC signal, and configured to provide the DC signal to the power storage device via two storage connection lines, and wherein the second interface includes:
at least one switch disposed between the two module connection lines and the two storage connection lines; and
at least one switch controller configured to control the at least one switch to prevent the power storage device from being electrically connected to an opposite polarity of the power source.

2. The medical implant of claim 1, wherein the second interface comprises four switches disposed between at least one of the two interface connection lines and the two storage connection lines.

3. The medical implant of claim 2, wherein the controller is configured to cause all the switches to be open during a change in the polarity of the AC signal.

4. The medical implant of claim 3, wherein the controller is configured to open closed switches when a current through the closed switches is below a threshold level.

5. The medical implant of claim 3, wherein the controller is configured to open closed switches a predetermined time after the switches were closed.

6. The medical implant of claim 1, wherein the DC power source is a battery.

7. The medical implant of claim 1, wherein the DC storage device is a capacitor.

8. The medical implant of claim 7, wherein the medical implant is a cochlear implant.

9. An internal component of a medical implant system comprising:
a DC power source; and
a DC/AC converter adapted to convert a DC signal from the power source to an AC signal;
a DC power storage device; and
a circuit connected to the DC/AC converter via two interface connection lines, and connected to the power storage device by two storage lines, wherein the circuit is configured to convert the AC signal and a DC signal, the circuit comprising:
at least one switch disposed between the two module connection lines and the two storage connection lines; and
at least one switch controller for controlling the at least one switch such to prevent the power storage device from being electrically connected to an opposite polarity of the power source.

10. The internal component of claim 9, wherein the medical implant system is a cochlear implant system.

11. The internal component of claim 10, wherein the circuit comprises four switches disposed between at least one of the two interface connection lines and the two storage connection lines.

12. The internal component of claim 11, wherein the controller is configured to open closed switches when the polarity changes.

13. The internal component of claim 12 wherein, the controller is configured to open closed switches when a current through the closed switches is below a threshold level.

14. The internal component of claim 12, wherein the controller is configured to open closed switches a predetermined time after the switches were closed.

15. The internal component claim 10, wherein the DC power source is a battery.

16. The internal component of claim 15, wherein the DC storage device is a capacitor.

17. A method for transferring power from a power source to a power storage device of a medical implant, comprising:
converting, with a first interface, a direct current (DC) signal from the power source into an alternating current (AC) signal;
providing the AC signal to a second interface via two interface connection lines;
converting, with the second interface, the AC signal to a DC signal for deliver to a power storage device via two storage connection lines, wherein the second interface comprises at least one switch disposed between the interface connection lines and the storage connection lines; and
causing the at least one switch to open and close such that at no time is one of the output lines electrically connected to one of the input lines of opposite polarity.

18. The method of claim 17, further comprising:
opening the at least one switch prior to a change in polarity of the input AC signal such that the at least one switch is completely open when the polarity changes.

19. The method of claim 18, further comprising:
opening the at least one switch when a current through the at least one switch is below a threshold level.

20. The method of claim 17, further comprising:
opening the at least one switch a predetermined time after the at least one switch was closed.
